(19) United States
(12) Patent Application Publication
(10) Pub. No.: US 2007/0048247 A1
(43) Pub. Date: Mar. 1, 2007

(54) Deodorizing tablets

(75) Inventors: Stephanie M. Martin, Woodstock, GA (US); John Gavin MacDonald, Decatur, GA (US); Jaeho Kim, Roswell, GA (US)

Correspondence Address: DORITY & MANNING, P.A., POST OFFICE BOX 1449, GREENVILLE, SC, 29602-1449, US

(73) Assignee: Kimberly-Clark Worldwide, Inc.

(21) Appl. No.: 11/217,098

(22) Filed: Aug. 31, 2005

(51) Int. Cl. A61K 31/12; A61L 9/00

(52) U.S. Cl. 424/76.2; 514/680

(57) ABSTRACT

A tablet that is capable of reducing odor when added to a liquid is provided. The deodorizing tablet may be used in a wide variety of applications in which odor control is desired, such as in toilets, water treatment/sewage systems, well water, bedpans, and so forth. In addition to providing odor control, the tablet is generally soluble in the liquid so that its components are better able to contact malodorous compounds contained within the liquid. In this regard, the present inventors have discovered that certain quinone compounds are both odor inhibiting and also soluble in liquids (e.g., urine), and thus particularly useful in forming the deodorizing tablet of the present invention.

### BACKGROUND OF THE INVENTION

It is often desirable to eliminate or reduce the noxious odors associated with urine in toilets, bedpans, nursing homes, etc. In this regard, several techniques have been developed in an attempt to reduce such odors. For example, U.S. Patent Application Publication No. 2005/0049154 to Brady describes a scented tablet for placement in a toilet bowl. Upon dissolving, the tablet releases a fragrance over a short period of time. Unfortunately, however, such conventional techniques do not adequately solve the problem of odor control in that the fragrances only “mask” the urine odor and may quickly dissipate. As such, a need exists for a more effective technique of eliminating and/or reducing urine odor.

### SUMMARY OF THE INVENTION

In accordance with one embodiment of the present invention, a deodorizing tablet for application to a liquid (e.g., urine) is disclosed. The tablet is formed from a compressed powder that comprises an odor-reducing quinone compound, the compressed powder being at least partially soluble in the liquid. In accordance with another embodiment of the present invention, a method for reducing odor in a liquid is disclosed. The method comprises contacting a tablet with the liquid, wherein the tablet is formed from a compressed powder that comprises an odor-reducing quinone compound. The tablet is allowed to dissolve so as to release the odor-reducing quinone compound.

Other features and aspects of the present invention are discussed in greater detail below.

### DETAILED DESCRIPTION OF REPRESENTATIVE EMBODIMENTS

Reference now will be made in detail to various embodiments of the invention, one or more examples of which are set forth below. Each example is provided by way of explanation, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention cover such modifications and variations.

Generally speaking, the present invention is directed to a tablet that is capable of reducing odor when added to a liquid. The deodorizing tablet may be used in a wide variety of applications in which odor control is desired, such as in toilets, water treatment'sewage systems, well water, bedpans, and so forth. In addition to providing odor control, the tablet is generally soluble in the liquid so that its components are better able to contact malodorous compounds contained within the liquid. In this regard, the present inventors have discovered that certain quinone compounds are both odor inhibiting and also soluble in liquids (e.g., urine), and thus particularly useful in forming the deodorizing tablet of the present invention.

Quinones refer to a class of compounds that possess a quinoid ring, such as anthraquinones, naphthaquinones, benzoquinones, hydroquinones, and so forth. Anthraquinones, for instance, have the following general formula:

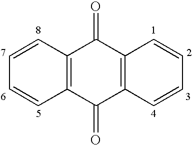

The numbers shown in the general formula represent a location on the fused ring structure at which substitution of a functional group may occur. Some examples of such functional groups that may be substituted on the fused ring structure include halogen groups (e.g., chlorine or bromine groups), sulfonyl groups (e.g., sulfonic acid salts), alkyl groups, benzyl groups, amino groups (e.g., primary, secondary, tertiary, or quaternary amines), carboxy groups, cyano groups, hydroxy groups, phosphorous groups, etc. Functional groups that result in an ionizing capability are often referred to as “chromophores.” Substitution of the ring structure with a chromophore causes a shift in the absorbance wavelength of the compound. Thus, depending on the type of chromophore (e.g., hydroxyl, carboxyl, amino, etc.) and the extent of substitution, a wide variety of quinones may be formed with varying colors and intensities. Other functional groups, such as sulfonic acids, may also be used to render certain types of compounds (e.g., higher molecular weight anthraquinones) water-soluble.

Anthraquinone compounds may be classified for identification by their Color Index (Cl) number, which is sometimes called a “standard.” For instance, some suitable anthraquinones that may be used in the present invention, as classified by their “Cl” number, include Acid Black 48, Acid Blue 25, D&C Green No. 5, Acid Blue 40, Acid Blue 41, Acid Blue 45, Acid Blue 80, Acid Blue 129, Acid Green 25, Acid Green 27, Acid Green 41, Acid Violet 43, Mordant Red 11 (Alizarin), Mordant Black 13 (Alizarin Blue Black B), Mordant Red 3 (Alizarin Red S), Mordant Violet 5 (Alizarin Violet 3R), Alizarin Complexone, Natural Red 4 (Carminic Acid), Disperse Blue 1, Disperse Blue 3, Disperse Blue 14, Natural Red 16 (Purpurin), Natural Red 8, Reactive Blue 2 (Procion Blue HB), Reactive Blue 19 (Remazol Brilliant Blue R); and so forth. Particularly useful anthraquinone compounds include, for instance, Acid Blue 25, Acid Blue 40, Acid Blue 45, Acid Blue 80, Acid Blue 129, Acid Green 25, Acid Green 27, Acid Green 41, D&C Green No. 5, Mordant Violet 5, Mordant Black 13, Reactive Blue 19, and Reactive Blue 2. The structures of Acid Blue 25, Acid Green 41, Acid Blue 45, Mordant Violet 5, Acid Blue 129, Acid Green 25, and Acid Green 27 are set forth below:

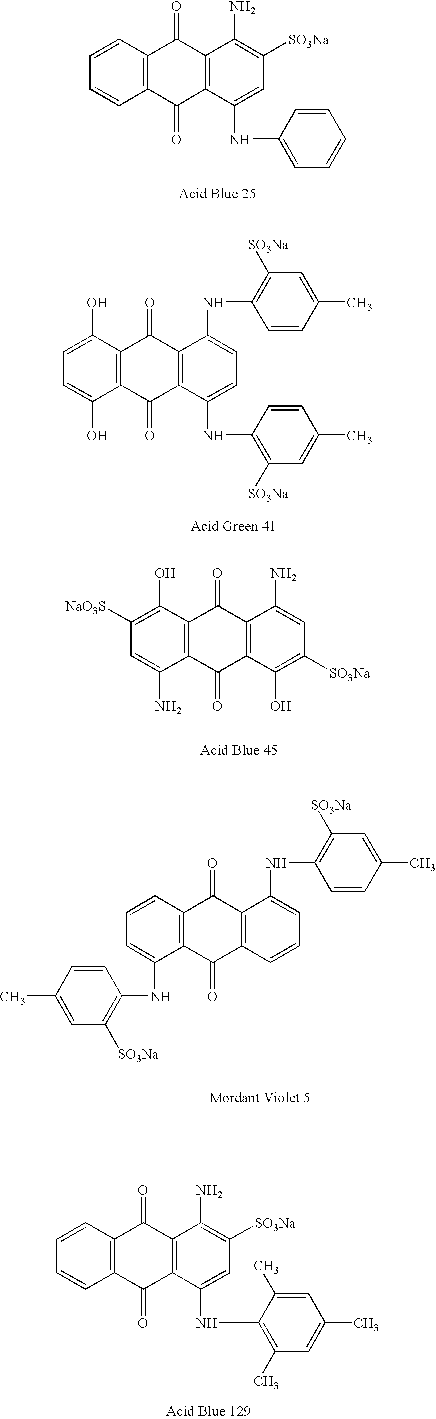

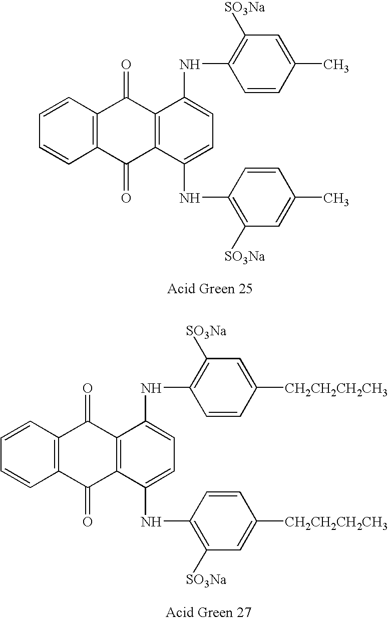

As stated above, other quinones may also be used in the present invention. For example, naphthaquinones may be used that have the following general formula:

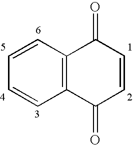

The locations 1-6 of the naphthaquinone compounds may be substituted 10 with functional groups in the manner described above. For instance, suitable examples of naphthaquinone compounds that may be used in the present invention include 1,4 naphthaquinone and 1,2 naphthaquinone, which have the following structures:

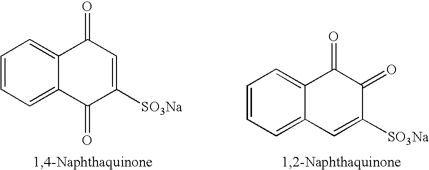

Besides their well-known ability to impart color, the present inventors have unexpectedly discovered that certain quinone compounds may also eliminate odor. Without intending to be limited by theory, it is believed that the odor caused by many compounds is eliminated by the transfer of electrons to and/or from the odorous compound. Specifically, electron reduction of odorous compounds via a reduction/oxidation (“redox”) reaction is believed to inhibit the production of the characteristic odor associated therewith. The surprising discovery that certain quinone compounds are able to eliminate odor is believed to be due their ability to function as an oxidizing agent in a redox reaction. Many common odorous compounds are capable of oxidizing (i.e., donate electrons) via a redox reaction. For instance, odorous compounds may include mercaptans (e.g., ethyl mercaptan), ammonia, amines (e.g., trimethylamine (TMA), triethylamine (TEA), etc.), sulfides (e.g., hydrogen sulfide, dimethyl disulfide (DMDS), etc.), ketones (e.g., 2-butanone, 2-pentanone, 4-heptanone, etc.) carboxylic acids (e.g., isovaleric acid, acetic acid, propionic acid, etc.), aldehydes, terpenoids, hexanol, heptanal, pyridine, and so forth. Upon oxidation, the odors associated with such compounds are often eliminated or at least lessened. It is also believed that the reduction of the quinone compound via the redox reaction is readily reversible, and thus the reduced quinone compound may be re-oxidized by any known oxidizing agent (e.g., oxygen, air, etc.). The reduction/oxidation reactions are rapid and may take place at room temperature. Thus, although the odor control mechanism may consume the quinone compounds, they may simply be regenerated by exposure to air. Thus, long-term odor control may be achieved without significantly affecting the ability of the quinone compound to impart the desired color.

The ability of quinone compounds to accept electrons from another substance (i.e., be reduced) may be quantified using a technique known as redox potentiometry. Redox potentiometry is a technique that measures (in volts) the affinity of a substance for electrons—its electronegativity—compared with hydrogen (which is set at 0). Substances more strongly electronegative than (i.e., capable of oxidizing) hydrogen have positive redox potentials. Substances less electronegative than (i.e., capable of reducing) hydrogen have negative redox potentials. The greater the difference between the redox potentials of two substances (ΔE), the greater the vigor with which electrons will flow spontaneously from the less positive to the more positive (more electronegative) substance. As is well known in the art, redox potential may be measured using any of a variety of commercially available meters, such as an Oxidation Reduction Potential (ORP) tester commercially available from Hanna Instruments, Inc. of Woonsocket, R. I. The redox potential of the quinone compounds may, for instance, be less than about −50 millivolts (mV), in some embodiments less than about −150 mV, in some embodiments less than about −300 mV, and in some embodiments, less than about −500 mV. Although not always the case, the redox potential may vary based on the number and location of functional groups, such as sulfonic acid, on the quinone structure. For example, 2-sulfonic acid anthraquinone has a redox potential of −380 mV; 2,6-disulfonic acid anthraquinone has a redox potential of −325 mV; and 2,7-disulfonic acid anthraquinone has a redox potential of −313 mV. Likewise, 2-sulfonic acid naphthaquinone has a redox potential of −60 mV. The use of other functional groups may also have an affect on the ultimate redox potential of the compound. For example, Acid Blue 25, which also contains amino- and aramid functional groups, has a redox potential of −605 mV.

In addition to their ability to oxidize odorous compounds, the present inventors have also discovered that the chemical structure of certain quinone compounds results in improved odor elimination. For example, anthraquinone compounds that have at least one unsubstituted ring may result in better odor inhibition than those that are substituted at each ring with a functional group. Interestingly, anthraquinone compounds that are unsubstituted at the “first” ring (i.e., positions 5 through 8) appear to be particularly effective in reducing odor. Suitable examples of anthraquinone compounds that are unsubstituted at locations at their first ring include, but are not limited to, Acid Blue 25, Acid Blue 129, Acid Green 25, and Acid Green 27, the structures of which are set forth above.

The quinone compound is provided in the form of a powder to aid in formation of the deodorizing tablet. The powder may be prepared using any conventional technique known in the art. For example, the powder may be prepared by drying the quinone compound in an oven and then converting the dried material to a powder using a milling device, such as a ball mill, bead mill, vibratory mill, sand mill, colloid mill, etc. Suitable dispersing agents may be, for example, condensation products of naphthalene sulfonic acid and formaldehyde, lignosulfonates or nonionic and anionic surface-active compounds. The resulting powder generally has an average particle size of from about 0.01 microns to about 20 microns, in some embodiments from about 0.5 microns to about 10 microns, and in some embodiments, from about 0.03 microns to about 6 microns. As used herein, the average size of a particle refers to its average length, width, height, and/or diameter. Some suitable anthraquinone powders are commercially available from Noveon Hilton Davis, Inc. of Cincinnati, Ohio; Sigma-Aldrich Chemical Co., Inc. of St. Louis, Mo.; and Acros Organics of Geel, Belgium.

The deodorizing tablet of the present invention may be formed from a powder using any of a variety of known techniques, such as wet granulation, dry granulation and/or direct compression. Wet granulation, for instance, involves blending and granulating of one or more components. Blending may be conducted, for instance, in a Banbury mixer, kneader, roll, single-screw or double-screw extruder, etc. Likewise, granulation may be conducted using a fluidized bed granulator, stirring granulator, tumbling granulator, tumbling fluidized bed granulator, extrusion granulator, etc. After granulation, the wet granulates are sieved, dried, and ground prior to compressing the tablet. In dry granulation, the quinone powder is compacted to yield a hard slug that is then ground and sieved before the addition of other ingredients and final compression. Compaction enhances particle size by aggregating the particles under high pressure (e.g., “compaction”). Although wet and dry granulation techniques are suitable, it is often desirable to form the deodorizing tablet of the present invention using a direction compression technique due to its simplicity and efficiency. More specifically, direct compression involves mixing together the ingredients and then directly compressing the resulting mixture. Any suitable compression molding machinery may be employed in such techniques, such as a tabletting (e.g., rotary, single-shot, double-shot or triple-shot type) or briquetting machine.

Regardless of the technique selected, a binder may be employed in some embodiments to facilitate the formation of granulates into a deodorizing tablet. The binder may be added in “dry” form to the powder blend or as a solution in a solvent (e.g., alcohol or water). Commonly used binders include polyvinylpyrrolidone, microcrystalline cellulose, sucrose, lactose, dextrose, sorbitol, mannitol, etc. One particularly suitable class of binders includes polysaccharides and derivatives thereof. Polysaccharides are polymers containing repeated carbohydrate units, which may be cationic, anionic, nonionic, and/or amphoteric. In one particular embodiment, the polysaccharide is a nonionic, cationic, anionic, and/or amphoteric cellulosic ether. Suitable nonionic cellulosic ethers may include, but are not limited to, alkyl cellulose ethers, such as methyl cellulose and ethyl cellulose; hydroxyalkyl cellulose ethers, such as hydroxyethyl cellulose, hydroxypropyl cellulose, hydroxypropyl hydroxybutyl cellulose, hydroxyethyl hydroxypropyl cellulose, hydroxyethyl hydroxybutyl cellulose and hydroxyethyl hydroxypropyl hydroxybutyl cellulose; alkyl hydroxyalkyl cellulose ethers, such as methyl hydroxyethyl cellulose, methyl hydroxypropyl cellulose, ethyl hydroxyethyl cellulose, ethyl hydroxypropyl cellulose, methyl ethyl hydroxyethyl cellulose and methyl ethyl hydroxypropyl cellulose; and so forth.

The mixture used to form the deodorizing tablet may contain various other additives known in the art to assist in a compression mold process. For example, inert fillers may be used as a bulking agent to decrease the concentration of a powder in the final tablet. Examples of such fillers may include silica, alumina, zirconia, magnesium oxide, titanium dioxide, iron oxide, zinc oxide, copper oxide, organic compounds such as polystyrene, and combinations thereof. In addition, a lubricant may be employed in the tablet and/or in the compression equipment to facilitate the release of the tablet. Disintegrants may also be employed to help the tablet disintegrate when placed in the desired liquid environment. The disintegration properties are primarily based on the ability of the disintegrant to swell in the presence of a fluid, such as water or urine. This swelling disrupts the continuity of the tablet structure, and thus allows the different components to enter into solution or into suspension. Commonly used disintegrants include native starches, modified starches, modified celluloses, microcrystalline cellulose or alginates.

Generally speaking, the amount of quinone powder contained within a deodorizing tablet may vary based on the level of odor control and optional color pattern or design utilized. For instance, in some embodiments, the quinone powder may comprise from about 10 wt. % to about 90 wt. %, in some embodiments from about 15 wt. % to about 80 wt. %, and in some embodiments, from about 20 wt. % to about 60 wt. %, of the tablet. Likewise, the amount of binder contained within a tablet may vary based on the compression technique employed and on the intended application. For instance, in some embodiments, the binder may comprise from about 0.1 wt. % to about 30 wt. %, in some embodiments from about 1 wt. % to about 20 wt. %, and in some embodiments, from about 2 wt. % to about 10 wt. %, of the tablet. When utilized, other ingredients, such as disintegrants, lubricants, etc., are typically employed in an amount from about 0.01 to about 20 wt. %, and in some embodiments, from about 0.1 wt. % to about 10 wt. % of the tablet.

The shape and size of the resulting deodorizing tablet may generally vary as is well known to those skilled in the art. If desired, the shape of the tablet may be selected so that the tablet better maintains its structure to during transportation, such as circular, columnar, elliptic columnar, conical, spherical, ellipsoidal, oval, masekku, disk-like, cubic, prism-like. A circular tablet, for example, may have a diameter of from about 1 to about 15 millimeters, and in some embodiments from about 1.5 to about 12 millimeters, as well as a length that is less than about 20 millimeters.

The ability of quinone compounds to reduce odor in accordance with the present invention is demonstrated by the following examples.

### EXAMPLE 1

The effectiveness of various quinone compounds in reducing urine odor was compared. Human female urine was initially collected and pooled by a nurse on staff at Kimberly-Clark Corporation. The pooled urine was added to mason jars in 50-milliliter aliquots using an automated pipette aid. Powders of Acid Blue 129 and Acid Green 27 (commercially available from Sigma-Aldrich Chemical Co., Inc. of St. Louis, Mo.), as well as D&C Green No. 5 (commercially available from Noveon Hilton Davis, Inc. of Cincinnati, Ohio), were then weighed and placed into the mason jars containing 50 milliliters of urine so that the final concentration of the powder was 8 millimolar (equivalent to 0.5 wt. %). The mason jars were placed in an incubator overnight at 37° C. and urine odor was assessed by a panel of individuals after 6 and 24 hours. A score of “10” was assigned to the most malodorous jar and a score of “1” was assigned to the least malodorous jar. The results are shown in Tables 1-2. The averages for each group are also set forth in Table 3.

TABLE 1
Odor rankings After 6 Hours

| | Ranking (number of panelists) | | | | | | | | | |
|---|---|---|---|---|---|---|---|---|---|---|
| Sample | 1 | 2 | 3 | 4 | 5 | 6 | 7 | 8 | 9 | 10 |
| Urine (control) | — | — | — | — | — | — | — | — | — | 5 |
| Acid Green 27 | — | — | — | 3 | 2 | — | — | — | — | — |
| D&C Green No. 5 | 1 | — | 3 | — | 1 | — | — | — | — | — |
| Acid Blue 129 | 4 | — | — | 1 | — | — | — | — | — | — |

TABLE 2
Odor rankings After 24 Hours

| | Ranking (number of panelists) | | | | | | | | | |
|---|---|---|---|---|---|---|---|---|---|---|
| Sample | 1 | 2 | 3 | 4 | 5 | 6 | 7 | 8 | 9 | 10 |
| Urine (control) | — | — | — | — | — | — | — | — | — | 6 |
| Acid Green 27 | 1 | — | 2 | — | — | — | 3 | — | — | — |
| D&C Green No. 5 | — | — | — | 3 | 1 | — | 2 | — | — | — |
| Acid Blue 129 | 5 | — | 1 | — | — | — | — | — | — | — |

TABLE 3
Average Odor rankings

| | |
|---|---|
| After 6 Hours | After 24 Hours |

| | Avg. | | Avg. |
|---|---|---|---|
| Sample | Ranking | Sample | Ranking |
| Urine | 10.0 | Urine | 10.0 |
| Acid Green 27 | 4.4 | Acid Green 27 | 4.5 |
| D&C Green No. 5 | 3.0 | D&C Green No. 5 | 5.2 |
| Acid Blue 129 | 1.6 | Acid Blue 129 | 1.3 |

As indicated, Acid Blue 129, D&C Green No. 5, and Acid Green 27 functioned effectively in reducing urine malodor.

### EXAMPLE 2

The effectiveness of various quinone compounds in reducing urine odor was compared. The powders chosen for this example were powders of Acid Blue 25, Acid Blue 45, Acid Blue 129, FD&C Blue No.1 (a triarylmethane), Acid Green 27, Acid Green 41, and Mordant Violet 5 (Alizarin Violet 3R) (commercially available from Sigma-Aldrich Chemical Co., Inc. of St. Louis, Mo.), as well as D&C Green No. 5 (commercially available from Noveon Hilton Davis, Inc. of Cincinnati, Ohio). Human female urine was initially collected and pooled by a nurse on staff at Kimberly-Clark Corporation. The pooled urine was added to mason jars in 50-milliliter aliquots using an automated pipette aid. Powders of the aforementioned quinone compounds were weighed and placed into the mason jars containing 50 milliliters of urine so that the final concentration of the powder was 1.6 millimolar (equivalent to 0.1 wt. %). The mason jars were placed in an incubator overnight at 37° C. and urine odor was assessed by a panel of individuals after 24 hours. A score of “10” was assigned to the most malodorous jar and a score of “1” was assigned to the least malodorous jar. The results from this study indicated that the 1.6-millimolar concentration of anthraquinone powders dissolved in urine were not optimal for the evaluation of odor control behavior.

Nevertheless, it was observed that Mordant Violet 5 did not perform as well in reducing odor as D&C Green No. 5. These quinone compounds are structural isomers, i.e., the sulfonic acid-containing phenyl rings are in a cis-conformation for the D&C Green No. 5 and in a trans- conformation for the Mordant Violet 5. Consequently, D&C Green No. 5 is substituted at positions 1 and 4 (the “second” anthraquinone ring), while Mordant Violet 5 is substituted at positions 1 and 5 (both the “first” and “second” anthraquinone rings). Without intending to be limited by theory, it is believed that the odor control properties of the quinone compound may be improved if positions 5 through 8 of the anthraquinone structure (the “first” anthraquinone ring) are unsubstituted.

### EXAMPLE 3

The effectiveness of various quinone compounds in reducing urine odor was compared. The quinone compounds chosen for this example were Acid Blue 25, Acid Blue 45, Acid Blue 129, FD&C Blue No.1 (triarylmethane), Acid Green 25, Acid Green 27, Acid Green 41, Mordant Violet 5 (Alizarin Violet 3R), 1,2 naphthaquinone-2-sulfonic acid potassium salt, and 1,4 naphthaquinone-2-sulfonic acid potassium salt. Human female urine was initially collected and pooled by a nurse on staff at Kimberly-Clark Corporation. The pooled urine was added to mason jars in 50-milliliter aliquots using an automated pipette aid. Powders of the aforementioned quinone compounds (commercially available from Sigma-Aldrich Chemical Co., Inc. of St. Louis, Mo. and Noveon Hilton Davis, Inc. of Cincinnati, Ohio) were weighed and placed into the mason jars containing 50 milliliters of urine so that the final concentration of the powder was 8 millimolar (equivalent to 0.5 wt. %). The mason jars were placed in an incubator overnight at 37° C.

To aid in the assessment of the quinone compounds, the mason jars were divided into two groups for a morning assessment, and the best of both groups were compared in an afternoon assessment (after a total of 24 hours). In addition to ranking the jars from least to most malodorous (on a scale from 1 to 7, with 7 being the most malodors), panelists were also asked to judge whether there were secondary (non-urine) odors present and, if so, the extent that the secondary odors were unpleasant (on a scale from 1 to 5, with 5 being extremely unpleasant).

The results for the first grouping are set forth below in Tables 4 and 5.

TABLE 4
Odor Control Rankings (First Grouping)

| | Urine Odor Ranking | | | | | | | |
|---|---|---|---|---|---|---|---|---|
| | (number of panelists) | | | | | | | Secondary Odor |
| Sample | 1 | 2 | 3 | 4 | 5 | 6 | 7 | Ranking |
| Pure Urine | — | — | 1 | — | — | 1 | 3 | — |
| Alizarin Violet 3R | — | | | 1 | — | 3 | 1 | 1 |
| 1,2 Naphthaquinone | — | 1 | 2 | 1 | — | — | 1 | 4, 5, 2 |
| Acid Blue 25 | 1 | 1 | 1 | 1 | 1 | — | — | 2 |
| Acid Green 25 | 3 | 1 | — | — | — | 1 | — | 1, 1, 4, 1, 1 |
| Acid Green 41 | 1 | 2 | 1 | 1 | — | — | — | 1 |
| FD&C Blue No. 1 | — | — | — | 1 | 4 | — | — | 3 |

TABLE 5
Average Urine Odor Ranking (First Grouping)

| Sample | Avg. Score |
|---|---|
| Pure Urine | 6.0 |
| Alizarin Violet 3R | 5.8 |
| 1,2 Naphthaquinone | 3.8 |
| Acid Blue 25 | 3.0 |
| Acid Green 25 | 2.2 |
| Acid Green 41 | 2.4 |
| FD&C Blue No. 1 | 4.8 |

In this first grouping, Acid Blue 25, Acid Green 25, and Acid Green 41 all functioned effectively to reduce odor. Although the majority of panelists felt that Acid Green 25 had a secondary odor, it was not found to be unpleasant by most panelists. That is, comments regarding this odor ranged from “slightly chemical”, “dirt-like”, “earthy”, or “damp.” In addition, 1,2-naphthaquinone also effectively reduced urine odor, although an unpleasant secondary smell was found to be present. The results for the second grouping are set forth below in Tables 6 and 7.

TABLE 6
Odor Control Rankings (Second Grouping)

| | Urine Odor Ranking | | | | | | |
|---|---|---|---|---|---|---|---|
| | (number of panelists) | | | | | | Secondary Odor |
| Sample | 1 | 2 | 3 | 4 | 5 | 6 | Ranking |
| Pure Urine | — | — | — | — | — | 5 | — |
| Acid Blue 45 | — | 1 | 2 | 1 | 1 | — | 4, 4 |
| 1,4 Naphthaquinone | 2 | 2 | 1 | — | — | — | 5, 3, 5, 4 |
| Acid Blue 129 | 3 | — | 2 | — | — | — | 1, 3 |
| Acid Green 27 | — | 2 | — | 1 | 2 | — | 2 |
| D&C Green No. 5 | — | — | — | 3 | 2 | — | 4 |

TABLE 7
Average Urine Odor Ranking (Second Grouping)

| Sample | Avg. Score |
|---|---|
| Pure Urine | 6.0 |
| Acid Blue 45 | 3.4 |
| 1,4 Naphthaquinone | 1.8 |
| Acid Blue 129 | 1.8 |
| Acid Green 27 | 3.6 |
| Acid Green 25 | 4.4 |

In this second grouping, D&C Green No. 5 did not perform as well compared to the other quinone compounds. The naphthaquinone compound was again found to have an unpleasant secondary odor. Acid Green 25 and D&C Green 25 have the same structure, but a slightly different purity level, i.e., D&C Green No. 5 is 89% pure and Acid Green 25 is 75% pure. It is possible that the performance of Acid Green 25 (Group 1) is a result of impurities in the compound, such as those that are anthraquinone in nature. Alternatively, processing differences (e.g., treatments during purification) may also have an effect.

In the afternoon, panelists were asked to assess the top performers in both groupings as a single group. In this assessment, panelists were given instructions to rank the least malodorous jar as “1” and the most malodorous jar as “10,” remaining free to rank the other jars between 2-9. This type of ranking was 5 chosen to obtain an idea of how much separation existed between the best and second best compounds, as well as how much worse the most malodorous compound is from the second most malodorous compound. The results are shown in Tables 8-10 (Table 10 shows the average urine odor rankings ignoring statistical anomalies).

TABLE 8
Odor Control Rankings (After 24 Hours)

| | Urine Odor Ranking | | | | | | | | | | |
|---|---|---|---|---|---|---|---|---|---|---|---|
| | (number of panelists) | | | | | | | | | | Secondary Odor |
| Sample | 1 | 2 | 3 | 4 | 5 | 6 | 7 | 8 | 9 | 10 | Ranking |
| Pure Urine | — | — | — | — | — | — | — | 2 | 1 | 2 | 5 |
| Acid Blue 25 | — | — | — | 1 | — | — | 1 | — | 2 | 1 | 4 |
| Acid Green 25 | 3 | 1 | — | — | — | — | | — | — | 1 | 1, 1, 4, 1, 2 |
| Acid Green 41 | 1 | 2 | 1 | — | — | — | 1 | — | — | — | 1, 3, 2 |
| Acid Blue 129 | 1 | — | 1 | 1 | 1 | 1 | — | — | — | — | 4, 5 |
| Acid Green 27 | — | 1 | — | — | — | — | — | 2 | 1 | 1 | 4 |
| D&C Green No. 5 | — | 1 | — | 1 | — | — | 2 | — | — | 1 | 4 |

TABLE 9
Average Urine Odor Ranking

| Sample | Avg. Score |
|---|---|
| Pure Urine | 9.0 |
| Acid Blue 25 | 6.8 |
| Acid Green 25 | 3.0 |
| Acid Green 41 | 3.0 |
| Acid Blue 129 | 3.8 |
| Acid Green 27 | 6.4 |
| D&C Green No. 5 | 6.0 |

TABLE 10
Average Urine Odor Ranking (with statistical corrections)

| Sample | Avg. Score |
|---|---|
| Pure Urine | 9.0 |
| Acid Blue 25 | 8.8 |
| Acid Green 25 | 1.3 |
| Acid Green 41 | 2.0 |
| Acid Blue 129 | 3.8 |
| Acid Green 27 | 8.8 |
| D&C Green No. 5 | 7.0 |

As indicated, Acid Green 25, Acid Green 41 and Acid Blue 129 achieved the best odor reduction.

### EXAMPLE 4

The effectiveness of various quinone compounds in reducing urine odor was compared. The quinone compounds chosen for this example were 1-amino-4-hydroxyanthraquinone, Procion Blue HB, Solvent Blue 59, Solvent Green 3, Remazol Brilliant Blue B, and 1,4-dihydroxyanthraquinone. Human female urine was initially collected and pooled at Kimberly-Clark Corporation. The pooled urine was added to mason jars in 50-milliliter aliquots using an automated pipette aid. Powders of the aforementioned quinone compounds (commercially available from Sigma-Aldrich Chemical Co., Inc. of St. Louis, Missouri and Noveon Hilton Davis, Inc. of Cincinnati, Ohio) were weighed and placed into the mason jars containing 50 milliliters of urine so that the final concentration of the powder was 8 millimolar (equivalent to 0.5 wt. %). The mason jars were placed in an incubator overnight at 37° C. The experimental set up is set forth below in more detail in Table 11.

TABLE 11
Experimental Setup

| | Formula | | Color in | |
|---|---|---|---|---|
| Sample | Weight | Grams | Urine | Soluble? |
| Solvent Blue 59 | 294.36 | 0.117744 | Gray | No |
| Solvent Green 3 | 418.48 | 0.167392 | Gray | No |
| 1-Amino-4- | 239.23 | 0.095692 | Pink/purple | Yes |
| Hydroxyanthraquinone | | | | |
| 1,4-Dihydroxyanthra- | 240.21 | 0.096084 | Orange | Sparingly |
| quinone | | | | |
| Procion Blue HB | 840.1 | 0.33604 | Turquoise | Yes |
| Remazol Brilliant | 624.52 | 0.249808 | Bright Blue | Yes |
| Blue R | | | | |

To aid in the assessment of the quinone compounds, panelists were asked to rank the mason jars from least to most malodorous (on a scale from 1 to 7, with 7 being the most malodors). The results are set forth below in Tables 12 and 13.

TABLE 12
Odor Control Rankings

| | Urine Odor Ranking | | | | | | |
|---|---|---|---|---|---|---|---|
| | (number of panelists) | | | | | | |
| Sample | 1 | 2 | 3 | 4 | 5 | 6 | 7 |
| Solvent Blue 59 | | | 1 | | 1 | 1 | 1 |
| Solvent Green 3 | | 1 | 1 | 1 | | 1 | |
| 1-Amino-4-Hydroxyanthraquinone | | | 1 | | 2 | | 1 |
| 1,4-Dihydroxyanthraquinone | | 1 | | 1 | | 1 | 1 |
| Procion Blue HB | 2 | 1 | | 1 | | | 1 |
| Remazol Brilliant Blue R | 2 | 2 | | | | | |
| Urine | | | | | | | 4 |

TABLE 13
Average Rankings

| Sample | Avg. Score | St. Dev |
|---|---|---|
| Solvent Blue 59 | 5.25 | 1.7078251 |
| Solvent Green 3 | 3.75 | 1.7078251 |
| 1-Amino-4-Hydroxyanthraquinone | 5 | 1.6329932 |
| 1,4-Dihydroxyanthraquinone | 4.75 | 2.2173558 |
| Procion Blue HB | 3.5 | 2.6457513 |
| Remazol Brilliant Blue R | 1.5 | 0.5773503 |
| Urine | 7 | 0 |

As indicated, the Remazol Brilliant Blue R was the most effective in reducing urine odor.

### EXAMPLE 5

The effectiveness of various quinone compounds in reducing urine odor was compared as described in Example 4. The quinone compounds chosen for this example were Acid Blue 129, D&C Green No. 5, Acid Blue 80, Acid Blue 40, Acid Blue 45, Remazol Brilliant Blue R, Alizarin Complexone, Acid Green 25, Acid Green 41, Acid Green 27, Acid Blue 41, Alizarin Blue Black B, Procion Blue HB, and Acid Violet 43. Due to the large number of quinone compounds being screened, the study was divided into two groups, i.e., “A” and “B.” The experimental setup is set forth in more detail below in Table 14.

TABLE 14
Experimental Setup

| | Formula | Grams | | |
|---|---|---|---|---|
| Sample | Weight | (8 mM) | Color in Urine | Soluble? |
| Acid Blue 129 | 458.47 | 0.183388 | Muddy green | Mostly |
| D&C Green No. 5 | 622.57 | 0.249028 | Deep green | Yes |
| Acid Green 25 | 622.57 | 0.249028 | Deep green | Yes |
| Acid Green 41 | 654.57 | 0.261828 | Bright green | Yes |
| Acid Green 27 | 706.75 | 0.2827 | Deep green | Mostly |
| Acid Blue 41 | 487.46 | 0.194984 | Sapphire | Yes |
| Alizarin Blue | 610.52 | 0.244208 | Purplish-Red | Yes |
| Black B | | | | |
| Acid Blue 80 | 678.68 | 0.271472 | Bright, deep blue | Yes |
| Acid Blue 40 | 473.43 | 0.189372 | Navy | Yes |
| Procion Blue HB | 840.09 | 0.336036 | Turquoise | Yes |
| Acid Blue 45 | 474.32 | 0.189728 | Navy | Yes |
| Acid Violet 43 | 329.37 | 0.131748 | Purple | Yes |
| Remazol Brilliant | 624.51 | 0.249804 | Bright, deep blue | Yes |
| Blue R | | | | |
| Alizarin | 421.36 | 0.168544 | Brick red | Mostly |
| Complexone | | | | |

Assessments were conducted the following day by asking panelists to rate each jar from 1 (least urine malodor) to 8 (most urine malodor). The results are set forth below in Tables 15-18.

TABLE 15
Odor Control Rankings (Group A)

| | Urine Odor Ranking | | | | | | | |
|---|---|---|---|---|---|---|---|---|
| | (number of panelists) | | | | | | | |
| Sample | 1 | 2 | 3 | 4 | 5 | 6 | 7 | 8 |
| Acid Green 25 | | 1 | 2 | 1 | | | | |
| Acid Green 41 | | 1 | 1 | | 1 | | 1 | |
| Acid Green 27 | | | 1 | 2 | | 1 | | |
| Acid Blue 41 | | | | 1 | 2 | 1 | | |
| Alizarin Blue Black B | 4 | | | | | | | |
| Procion Blue HB | | | | | 1 | 1 | 1 | 1 |
| Acid Violet 43 | | 2 | | | | | 1 | 1 |
| Urine | | | | | | 1 | 1 | 2 |

TABLE 16
Average Rankings (Group A)

| Sample | Avg. Score | St. Dev. |
|---|---|---|
| Acid Green 25 | 3 | 0.816496581 |
| Acid Green 41 | 4.25 | 2.217355783 |
| Acid Green 27 | 4.25 | 1.258305739 |
| Acid Blue 41 | 5 | 0.816496581 |
| Urine | 7.25 | 0.957427108 |
| Alizarin Blue Black B | 1 | 0 |
| Procion Blue | 6.5 | 1.290994449 |
| Acid Violet 43 | 4.75 | 3.201562119 |

TABLE 17
Odor Control Rankings (Group B)

| | Urine Odor Ranking | | | | | | | |
|---|---|---|---|---|---|---|---|---|
| | (number of panelists) | | | | | | | |
| Sample | 1 | 2 | 3 | 4 | 5 | 6 | 7 | 8 |
| Acid Blue 129 | | 1 | | | 1 | 1 | 1 | |
| D&C Green No. 5 | | 1 | 1 | | 1 | | 1 | |
| Acid Blue 80 | | | | 1 | 1 | | | 2 |
| Acid Blue 40 | | 1 | | 2 | | | 1 | |
| Acid Blue 45 | 2 | | | | | 1 | 1 | |
| Remazol Brilliant Blue | 2 | | | 1 | | 1 | | |
| Alizarin Complexone | | 1 | 2 | | | | | 1 |
| Urine | | | 1 | | 1 | 1 | | 1 |

TABLE 18
Average Rankings (Group B)

| Sample | Avg. Score | St. Dev. |
|---|---|---|
| Acid Blue 129 | 5 | 2.160246899 |
| D&C Green | 4.25 | 2.217355783 |
| Acid Blue 80 | 6.25 | 2.061552813 |
| Acid Blue 40 | 4.25 | 2.061552813 |
| Acid Blue 45 | 3.75 | 3.201562119 |
| Remazol Brilliant Blue R | 3 | 2.449489743 |
| Alizarin Complexone | 4 | 2.708012802 |
| Urine | 5.5 | 2.081665999 |

From Group A, Alizarin Blue Black B performed best. For Group B, Remazol Brilliant Blue R, Acid Blue 45, Alizarin Complexone, Acid Blue 129, Acid Blue 40, and D&C Green No. 5 performed well.

### EXAMPLE 6

The effectiveness of various quinone compounds in reducing urine odor was compared as described in Example 5. The quinone compounds chosen for this example were Alizarin Blue Black B, Remazol Brilliant Blue R and Acid Green 25. The results are set forth below in Tables 19 and 20.

TABLE 19
Odor Control Rankings

| | Urine Odor Ranking | | | | | | | |
|---|---|---|---|---|---|---|---|---|
| | (number of panelists) | | | | | | | |
| Sample | 1 | 2 | 3 | 4 | 5 | 6 | 7 | 8 |
| D&C Green No. 5 | | | | | 2 | | 1 | 1 |
| Acid Blue 45 | | 1 | | | | 1 | 2 | |
| Remazol Brilliant Blue | | 1 | 1 | | | | 2 | |
| Alizarin Complexone | 1 | | 1 | 1 | | | | 1 |
| Acid Green 25 | 2 | 1 | | | 1 | | | |
| Acid Green 41 | | | 1 | 1 | | 1 | | 1 |
| Alizarin Blue Black B | 1 | | 2 | 1 | | | | |
| Urine | | | | | | 1 | | 2* |

*One panelist inadvertently gave a value of “10” rather “8.”

TABLE 20
Average Rankings

| Sample | Avg. Score | St. Dev |
|---|---|---|
| D&C Green | 6.25 | 1.5 |
| Acid Blue 45 | 5.5 | 2.380476143 |
| Remazol Brilliant Blue R | 4.75 | 2.62995564 |
| Alizarin Complexone | 4 | 2.943920289 |
| Urine | 8 | 1.632993162 |
| Acid Green 25 | 2.25 | 1.892969449 |
| Acid Green 41 | 5.25 | 2.217355783 |
| Alizarin Blue Black B | 2.75 | 1.258305739 |

### EXAMPLE 7

The effectiveness of various quinone compounds in reducing urine odor was compared. The quinone compounds chosen for this example were Acid Blue 129, D&C Green No. 5, Acid Green 25, Acid Green 41, Acid Green 27, Acid Blue 41, Alizarin Blue Black B, Acid Blue 80, Acid Blue 40, Procion Blue HB, Acid Blue 45, Acid Violet 43, Remazol Brillian Blue R, Alizarin Complexone, Carminic Acid, Emodin, Acid Black 48, and Acid Blue 25. Human female urine was initially collected and pooled at Kimberly-Clark Corporation. The pooled urine was added to mason jars in 50-milliliter aliquots using an automated pipette aid. Powders of the aforementioned quinone compounds (commercially available from Sigma-Aldrich Chemical Co., Inc. of St. Louis, Missouri and Noveon Hilton Davis, Inc. of Cincinnati, Ohio) were weighed and placed into the mason jars containing 50 milliliters of urine so that the final concentration of the powder was 16 millimolar (equivalent to 1 wt. %). The mason jars were placed in an incubator overnight at 37° C. The experimental setup is set forth in more detail below in Table 21.

TABLE 21
Experimental setup

| | Formula | Grams | | |
|---|---|---|---|---|
| Sample | Weight | (16 mM) | Color in Urine | Soluble? |
| Acid Blue 129 | 458.47 | 0.366776 | Muddy blue | Somewhat |
| D&C Green No. 5 | 622.57 | 0.498056 | Bright deep green | Yes |
| Acid Green 25 | 622.57 | 0.498056 | Bright deep green | Mostly |
| Acid Green 41 | 654.57 | 0.523656 | Bright green | Yes |
| Acid Green 27 | 706.75 | 0.565400 | Dark green | Mostly |
| Acid Blue 41 | 487.46 | 0.389968 | Sapphire | Yes |
| Alizarin Blue | 610.52 | 0.488416 | Grape | Yes |
| Black B | | | | |
| Acid Blue 80 | 678.68 | 0.542944 | Bright blue | Mostly |
| Acid Blue 40 | 473.43 | 0.378744 | Navy | Somewhat |
| Procion Blue HB | 840.09 | 0.672072 | Dark turquoise | Yes |
| Acid Blue 45 | 474.32 | 0.379456 | Navy | Mostly |
| Acid Violet 43 | 329.37 | 0.263496 | Muddy violet | No |
| Remazol Brillian | 624.51 | 0.499608 | Bright blue | Yes |
| Blue R | | | | |
| Alizarin | 421.36 | 0.337088 | Deep red/orange | No |
| Complexone | | | | |
| Carminic Acid | 492.39 | 0.393912 | Red | Yes |
| Emodin | 270.24 | 0.216192 | Orange | No |
| Acid Black 48 | 459.45 | 0.367560 | Blue/black | Somewhat |
| Acid Blue 25 | 616.49 | 0.493192 | Navy | Mostly |

Jars were wrapped in foil and placed in an incubator at 37° C. overnight. The jars were divided into four groups (C-F), each sharing the same control jar of urine alone (no powder). Assessments were conducted the following day by asking panelists to rate each jar from 1 (least urine malodor) to 10 (most urine malodor). The results are provided in Tables 22-29.

TABLE 22
Odor Control Rankings (Group C)

| | Urine Odor Ranking | | | | | | | | | |
|---|---|---|---|---|---|---|---|---|---|---|
| | (number of panelists) | | | | | | | | | |
| Sample | 1 | 2 | 3 | 4 | 5 | 6 | 7 | 8 | 9 | 10 |
| Acid Black 48 | | 1 | | | | 1 | | 1 | | 2 |
| Acid Blue 25 | 3 | | | 1 | 1 | | | | | |
| Acid Blue 41 | | 2 | 2 | | | | 1 | | | |
| Emodin | | | | | 1 | | | 2 | 2 | |
| Alizarin Blue Black B | 1 | | 2 | 2 | | | | | | |
| Urine Control | | | | | | | | 1 | | 4 |

TABLE 23
Average Rankings (Group C)

| Sample | Avg. Score | St. Dev |
|---|---|---|
| Acid Black 48 | 7.2 | 3.34664 |
| Acid Blue 25 | 2.4 | 1.949359 |
| Acid Blue 41 | 3.4 | 2.073644 |
| Emodin | 7.8 | 1.643168 |
| Alizarin Blue Black B | 3 | 1.224745 |
| Urine Control | 9.6 | 0.894427 |

TABLE 24
Odor Control Rankings (Group D)

| | Urine Odor Ranking | | | | | | | | | |
|---|---|---|---|---|---|---|---|---|---|---|
| | (number of panelists) | | | | | | | | | |
| Sample | 1 | 2 | 3 | 4 | 5 | 6 | 7 | 8 | 9 | 10 |
| Alizarin Complexone | | | | 1 | 1 | | 2 | | | 1 |
| Acid Green 41 | 4 | | 1 | | | | | | | |
| D&C Green No. 5 | | | | | 2 | | 1 | | 2 | |
| Alizarin Violet 43 | 1 | | 1 | 1 | 1 | | | 1 | | |
| Urine Control | | | | | | | | | 1 | 4 |

TABLE 25
Average Rankings (Group D)

| Sample | Avg. Score | St. Dev |
|---|---|---|
| Alizarin Complexone | 6.6 | 2.302173 |
| Acid Green 41 | 1.4 | 0.894427 |
| D&C Green No. 5 | 7 | 2 |
| Alizarin Violet 43 | 4.2 | 2.588436 |
| Urine Control | 9.8 | 0.447214 |

TABLE 26
Odor Control Rankings (Group E)

| | Urine Odor Ranking | | | | | | | | | |
|---|---|---|---|---|---|---|---|---|---|---|
| | (number of panelists) | | | | | | | | | |
| Sample | 1 | 2 | 3 | 4 | 5 | 6 | 7 | 8 | 9 | 10 |
| Carminic Acid | 2 | | 1 | 1 | | | | | 1 | |
| Acid Green 27 | 1 | | | | 1 | | 1 | 1 | | 1 |
| Remazol Brilliant Blue R | 1 | 2 | 1 | | | | | | 1 | |
| Acid Blue 129 | 1 | | | 1 | | | 1 | | 2 | |
| Acid Blue 45 | | | 2 | | | | | 2 | | 1 |
| Urine Control | | | 1 | | | | | | | 4 |

TABLE 27
Average Rankings (Group E)

| Sample | Avg. Score | St. Dev |
|---|---|---|
| Carminic Acid | 3.6 | 3.286335 |
| Acid Green 27 | 6.2 | 3.420526 |
| Remazol Brilliant Blue R | 3.4 | 3.209361 |
| Acid Blue 129 | 6 | 3.464102 |
| Acid Blue 45 | 6.4 | 3.209361 |
| Urine Control | 8.6 | 3.130495 |

TABLE 28
Odor Control Rankings (Group F)

| | Urine Odor Ranking | | | | | | | | | |
|---|---|---|---|---|---|---|---|---|---|---|
| | (number of panelists) | | | | | | | | | |
| Sample | 1 | 2 | 3 | 4 | 5 | 6 | 7 | 8 | 9 | 10 |
| Acid Blue 80 | 1 | | | | 1 | 1 | 1 | 1 | | |
| Acid Green 25 | 2 | 1 | | | | 2 | | | | |
| Acid Blue 40 | 1 | 1 | 1 | 1 | | | | | 1 | |
| Procion Blue HB | 1 | | | 1 | 1 | 1 | | | 1 | 1 |
| Urine Control | | | | | 1 | | | | | 4 |

TABLE 29
Average Rankings (Group F)

| Sample | Avg. Score | St. Dev |
|---|---|---|
| Acid Blue 80 | 5.4 | 2.701851 |
| Acid Green 25 | 3.2 | 2.588436 |
| Acid Blue 40 | 3.8 | 3.114482 |
| Procion Blue HB | 5.2 | 3.271085 |
| Urine Control | 9 | 2.236068 |

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A deodorizing tablet for application to a liquid, wherein the tablet is formed from a compressed powder that comprises an odor-reducing quinone compound, the compressed powder being at least partially soluble in the liquid.

2. The deodorizing tablet of claim 1, wherein the odor-reducing quinone compound is selected from the group consisting of anthraquinones, naphthaquinones, benzoquinones, hydroquinones, and combinations thereof.

3. The deodorizing tablet of claim 1, wherein the odor-reducing quinone compound is an anthraquinone having the following structure: 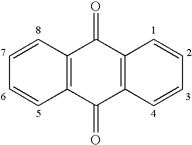
wherein the numbers 1 through 8 refer to optional substitution positions for functional groups.

4. The deodorizing tablet of claim 3, wherein the anthraquinone is substituted with halogen groups, alkyl groups, benzyl groups, amino groups, carboxy groups, cyano groups, hydroxy groups, phosphorous groups, sulfonic acid groups, or combinations thereof.

5. The deodorizing tablet of claim 3, wherein at least one ring of the anthraquinone is unsubstituted with functional groups.

6. The deodorizing tablet of claim 5, wherein positions 5 through 8 of the anthraquinone are unsubstituted with functional groups.

7. The deodorizing tablet of claim 1, wherein the odor-reducing quinone compound is selected from the group consisting of Acid Blue 25, Acid Blue 40, Acid Blue 45, Acid Blue 80, Acid Blue 129, Acid Green 25, Acid Green 27, Acid Green 41, D&C Green No. 5, Mordant Violet 5, Mordant Black 13, Reactive Blue 19, and Reactive Blue 2.

8. The deodorizing tablet of claim 1, wherein the powder has an average particle size of from about 0.01 to about 20 microns.

9. The deodorizing tablet of claim 1, wherein the powder comprises from about 10 wt. % to about 90 wt. % of the tablet.

10. The deodorizing tablet of claim 1, wherein the powder comprises from about 20 wt. % to about 60 wt. % of the tablet.

11. The deodorizing tablet of claim 1, wherein the tablet further comprises a binder.

12. The deodorizing tablet of claim 11, wherein the binder comprises from about 0.1 wt. % to about 30 wt. % of the tablet.

13. The deodorizing tablet of claim 1, wherein the powder consists essentially of the odor-reducing quinone compound.

14. The deodorizing tablet of claim 1, wherein the liquid is urine.

15. A method for reducing odor in a liquid, the method comprising:
contacting a tablet with the liquid, wherein the tablet is formed from a compressed powder that comprises an odor-reducing quinone compound; and
allowing the tablet to dissolve in the liquid so as to release the odor-reducing quinone compound.

16. The method of claim 15, wherein the odor-reducing quinone compound is an anthraquinone having the following structure: 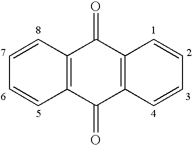
wherein the numbers 1 through 8 refer to optional substitution positions for functional groups.

17. The method of claim 16, wherein the anthraquinone is substituted with halogen groups, alkyl groups, benzyl groups, amino groups, carboxy groups, cyano groups, hydroxy groups, phosphorous groups, sulfonic acid groups, or combinations thereof.

18. The method of claim 16, wherein at least one ring of the anthraquinone is unsubstituted with functional groups.

19. The method of claim 18, wherein positions 5 through 8 of the anthraquinone are unsubstituted with functional groups.

20. The method of claim 15, wherein the odor-reducing quinone compound is selected from the group consisting of Acid Blue 25, Acid Blue 40, Acid Blue 45, Acid Blue 80, Acid Blue 129, Acid Green 25, Acid Green 27, Acid Green 41, D&C Green No. 5, Mordant Violet 5, Mordant Black 13, Reactive Blue 19, and Reactive Blue 2.

21. The method of claim 15, wherein the powder comprises from about 10 wt. % to about 90 wt. % of the tablet.

22. The method of claim 15, wherein the powder comprises from about 20 wt. % to about 60 wt. % of the tablet.

23. The method of claim 15, wherein the tablet further comprises a binder.

24. The method of claim 24, wherein the binder comprises from about 0.1 wt. % to about 30 wt. % of the tablet.

25. The method of claim 15, wherein the powder consists essentially of the odor-reducing quinone compound.

26. The method of claim 15, wherein the liquid is urine.
